(19) United States
(12) Patent Application Publication
(10) Pub. No.: US 2011/0207823 A1
(43) Pub. Date: Aug. 25, 2011

(54) 12-HOUR SUSTAINED-RELEASE METOCLOPRAMIDE

(75) Inventors: John Claude Savoir Vilboeuf, Mexico City (MX); Maria Teresa de Jesus Francisco Doce, Mexico City (MX); Teresita del Niño Jesús Costales González, Mexico City (MX); Miriam Villa Vargas, Mexico de Nezahualcoyotl Estado (MX)

(73) Assignee: POSIVISIONARY SOLUTIONS LLP, Bromley, Kent (GB)

(21) Appl. No.: 12/935,191

(22) PCT Filed: Mar. 27, 2009
(86) PCT No.: PCT/MX2009/000025

§ 371 Date: Oct. 20, 2010

(30) Foreign Application Priority Data
Mar. 28, 2008 (MX) MX/A/2018/004267

(51) Int. Cl. A61K 31/166; A61P 1/08; A61P 1/04

(52) U.S. Cl. 514/619; 424/468

(57) ABSTRACT

The present invention consists of an extended-release metoclopramide hydrochloride pharmaceutical composition, in 15 mg drug substance tablets, for use in gastrointestinal disorders. The formulation is mainly composed of a hydrophilic polymer, a hydrophobic polymer, a hydrophilic component and metoclopramide hydrochloride. The hydrophilic polymer is swollen by hydration when contacting water, forming a gel coat which controls drug substance release. The water inside the matrix dissolves the drug substance and this is diffused outside through the gel coat. The hydrophobic polymer shows plastic deformation properties under compression, tending to surround the drug substance particles reducing the pore quantity and dimensions in the matrix structure, delaying as a consequence the drug substance release. The hydrophilic component is part of the gel coating structure providing support thereto. Drug substance is the metoclopramide hydrochloride or a pharmaceutically acceptable salt thereof.

### FIELD OF INVENTION

The present invention consists of an extended-5 release metoclopramide hydrochloride pharmaceutical composition, in 100 milligram tablets, containing about 15 milligrams of drug substance, for use in gastrointestinal disorders.

### BACKGROUND OF INVENTION

Gastrointestinal prokinetics promote or enhance the intestine wall contraction coordination, elicit an increase in propulsive motility and as a consequence, a displacement of its contents.

Currently, prokinetics are the selected drug products for treatment of motor disorders in gastrointestinal tract, such as those associated with gastroesophageal reflux, chronic dyspepsia, gastroparesis and acute or idiopathic intestinal pseudo obstruction (Tonini, M. 1996, Pharmacol. Res. Vol. 33:217-226).

Metoclopramide is found among the most widely used prokinetics. Metoclopramide is a benzamide derivative, structurally related with procainamide and sulpiride. As this last compound, it shows antagonistic activity with dopamine, with a selective affinity with D-2 receptors.

Its behavioral, motor and neuroendocrine effects have been related with its antidopaminergic activity. Metclopramide has antiemetic effects which assumedly are a result of its action in chemorecept or triggering zone. Metoclopramide increases the rest pressure in lower esophageal sphincter, and causes an increase in amplitude of peristaltic contractions in esophagus, gastric antrum and small intestine. Said effects result in esophageal evacuation increase, accelerated gastric emptying and decreased transit through small intestine. These effects are blocked by atropine and opioids, but not by vagotomy.

Metoclopramide increases the tone and amplitude of gastric contractions, relaxing the pyloric sphincter and duodenal bulb and increases the duodenum and jejunum peristalsis causing an increase in velocity of said gastric and intestinal emptying. Metoclopramide raises prolactin present in serum and causes transient increases in aldosterone circulating levels. It is thought that said effects are due to a dopamine receptor blockage on cellular adrenocortical and pituitary gland level. Metoclopramide does not stimulate gastric acid secretion. In medical practice, metoclopramide is used in gastroparesis treatment, in order to decrease the disturbances associated with gastroenterological explorations, nausea and vomit which are frequent after surgical interventions, and with esophageal reflux. Injectable form of this compound is used to ease small intestine intubation and barium passage through intestine in radiology procedures. Metoclopramide tablets are used in treatment of symptoms associated with gastroparesis in diabetic patients. Gastroparesis symptoms include nausea and vomit, an early sensation of satiety and abdominal disturbances. Treatment options for gastroparesis include diet, behavioral changes, prokinetic drugs and surgical interventions (Akheel, S. A. Rattansingh & S Furtado. J. Postgrad. Med. 2005. Vol. 51, No. 1: 54-60). Gastroparesis is an abnormal gastric motility condition characterized by a slow gastric emptying in absence of any mechanical obstruction, normally occurring in persons suffering of diabetes. This drug product has also shown its usefulness in treatment of vomit caused by several factors (Ponte, C D., and J. M. Nappi, 1981, American J Hosp. Pharm. Vol. 38, No. 6:829-833).

Metoclopramide has been used in treatment of esophageal reflux, one of the most frequent diseases in gastroenterological practice. This disease is a retrograde movement of gastric contents through the lower esophageal sphincter towards esophagus. Symptoms associated with esophageal reflux include a 5 strong chest heartburn, acidic regurgitation, non-heart chest pain, dysphagia, globose pharyngitis, chronic cold, asthma, laryngitis, chronic sinusitis and tooth decay {Starr, M., Meining, A & D. Allescher. 2000. Digestive Diseases. Vol. 18: 93-10 102). The use of metoclopramide in treatment of post-surgery nausea and vomit is particularly important, relevant morbidity causes after anesthesia and surgery (Domino, K., et al. 1999. Anesth. Analg. Vol. 88: 1370-1379).

In spite of the beneficial effects resulting from this drug product in gastrointestinal disorders such as those above mentioned, there are studies (Bateman, D. N., et al. 1979. Br. J. Pharmacol. Vol. 8: 179-182) which report undesirable side effects, for instance, akathisia (inability to remain quietly seated), due to the administration of this drug in immediate release dosages, where metoclopramide peaks in plasma reach above 100 nanograms per milliliter.

Other reports on the adverse effects after administering this compound disclose that long-term use of this compound in immediate-release dosages caused appearance of psychosis in patients (Lu, M. L., et al. 2002. Ann Pharmacotherapy. Vol. 36, No. 9: 1387-1390).

Appearance of extrapyramidal effects (involuntary slow movements, Huntington's disease) collateral to the use of immediate-release metoclopramide, such as parkinsonism induction have also been reported {Sirota, R. A., et al. 1986. Arch. Int Med. Vol. 146, 10 No. 10: 2070-2071).

Salazar et al (Salazar, A. B., et al 2005, Neurologia, Neurocirugia y Psiquiatría, Vol. 38, No. 1: 1-6), disclose the appearance of extrapyramidal effects, anxiety and depression at higher doses. Side effects such as sleepiness, drowsiness and anxiety are also frequent.

Following are references of a number of patents which are related with the invention:

U.S. Pat. No. 4,656,024 consists of a slow release 20 mg metoclopramide pharmaceutical composition, having a first metoclopramide coating from 1 to 20% by weight of metoclopramide, from 0.01 to 0.5% by weight of stearic acid and 5 to 15% by weight of talc, and from 2% to 10% by weight of silica gel and sequential coatings of shellac and raethacrylate polymer as semi-permeable membrane, being shellac coating from 1 to 10% by weight in total composition.

U.S. Pat. No. 4,780,322 consisting of a slow release metoclopramide pharmaceutical composition, containing sulfonated resins and carboxylic resins. U.S. Pat. No. 4,808,416 of slow release, sequentially consists of a metoclopramide pharmaceutical composition wherein said drug substance is located in a core; a first ethylacrylate and methylmethacrylate copolymer coating and a second hydroxypropylmethylphtalate cellulose enteric coating.

U.S. Pat. No. 6,770,262 is referred to a method for treatment of gastroparesis, using nasally metoclopramide.

U.S. patent application 2005/0282873 refers to a controlled-release pharmaceutical composition with metoclopramide as drug substance and a hydrophilic polymer, specifically xanthan gum.

Traditionally metoclopramide is found in an immediate-release dosage form, which requires a delivery every 8 hours. This dosage form in addition of being complex for patient, involves the risks of reaching plasm concentrations which cause extrapyramidal effects.

One of the objects of present invention is to provide a compound of metoclopramide hydrochloride, or a pharmaceutically acceptable extended release salt thereof, with a lower delivery frequency.

Another object of present invention is to provide a compound of metoclopramide hydrochloride, or a pharmaceutically acceptable extended release salt thereof, which may be administered every 12 hours.

A further objective of present invention is to provide a compound of metoclopramide hydrochloride, or a pharmaceutically acceptable salt thereof, in such a way that being effective but without reaching plasma concentrations which cause extrapyramidal effects.

### BRIEF DESCRIPTION OF THE DRAWINGS

FIG. 1 shows a mean velocity dissolution profile for metoclopramide hydrochloride controlled-release tablets.

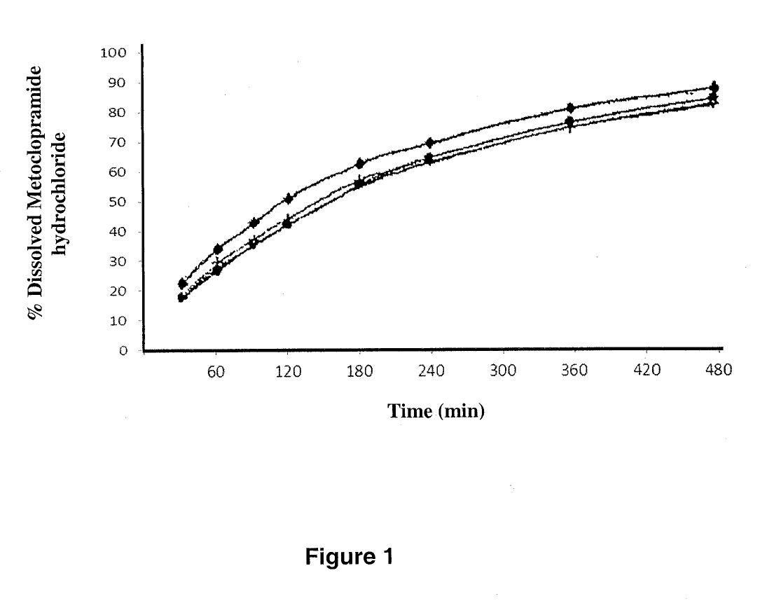

FIG. 2 shows a slow velocity dissolution profile for metoclopramide hydrochloride controlled-release tablets.

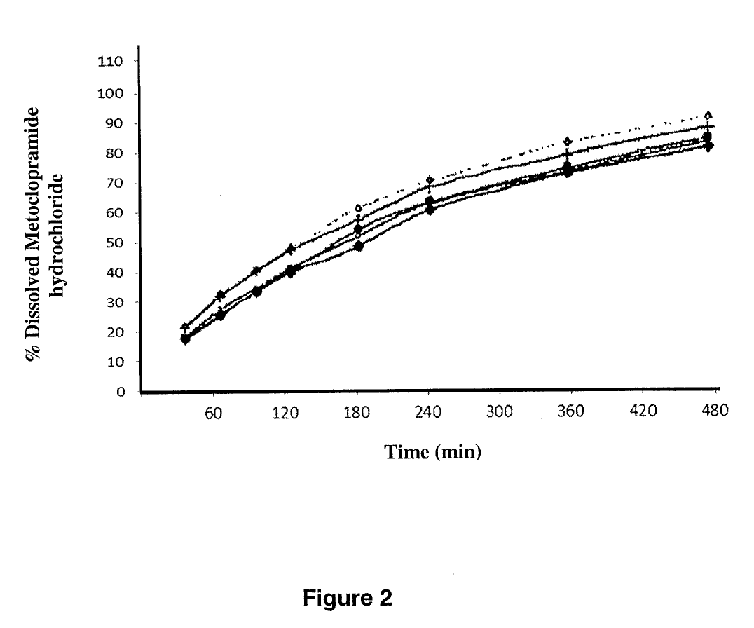

FIG. 3 shows a high velocity dissolution profile for metoelopramide hydrochloride controlled-release tablets.

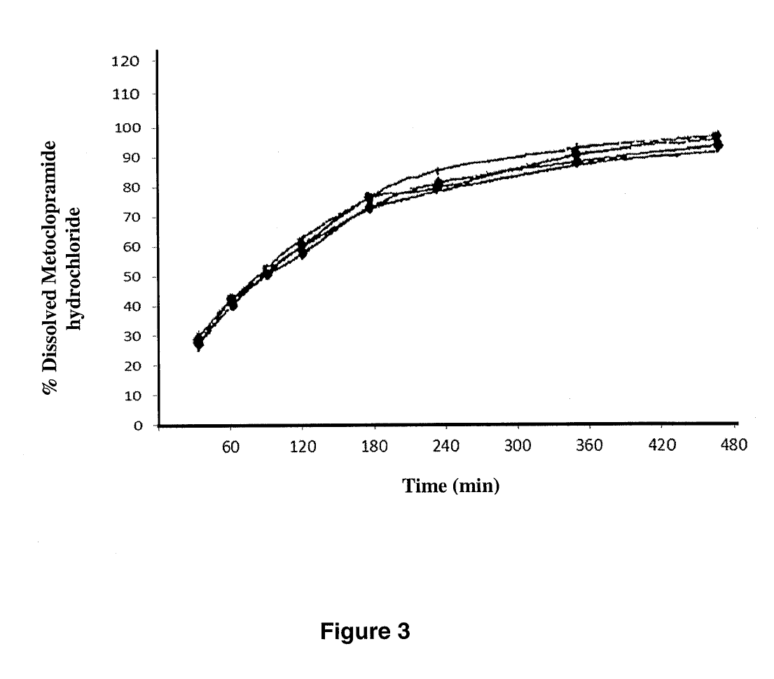

FIG. 4 shows a comparison of dissolution profiles for three formulations.

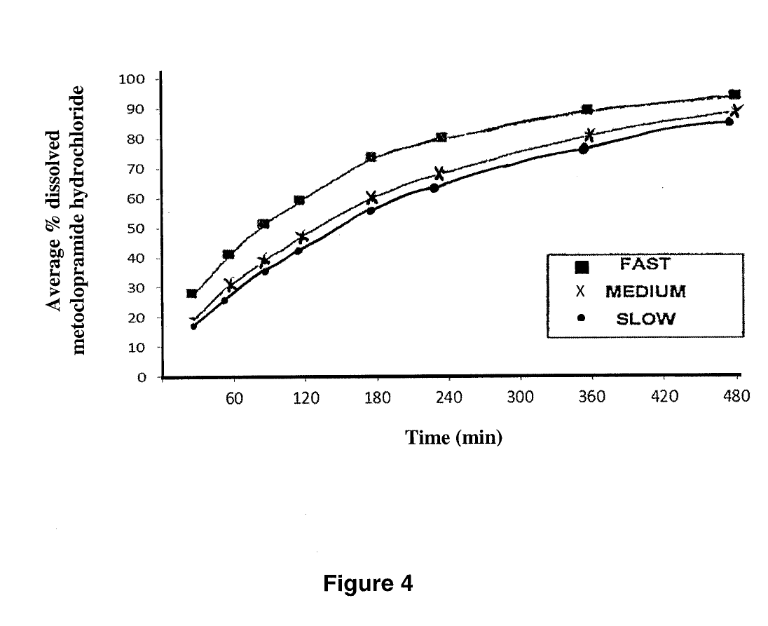

FIG. 5 shows dissolution profiles for metoclopramide hydrochloride extended- or immediate-release tablets.

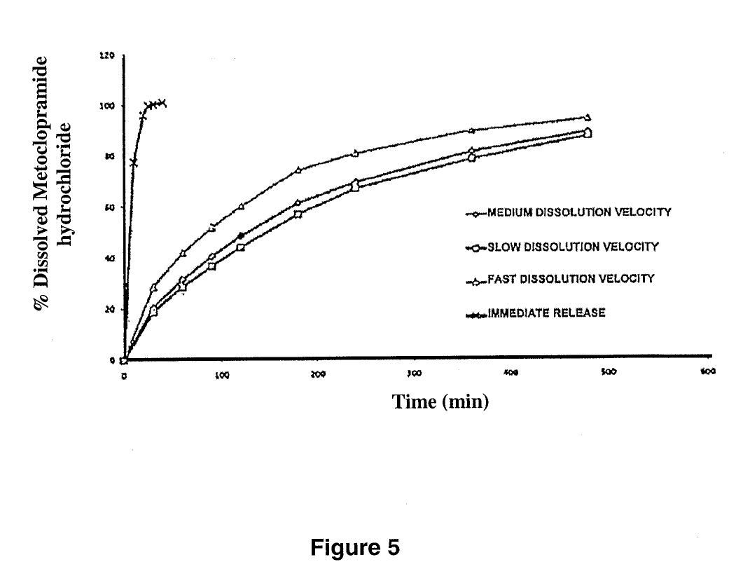

FIG. 6 shows the plasma profiles for tablets at several dissolution velocities.

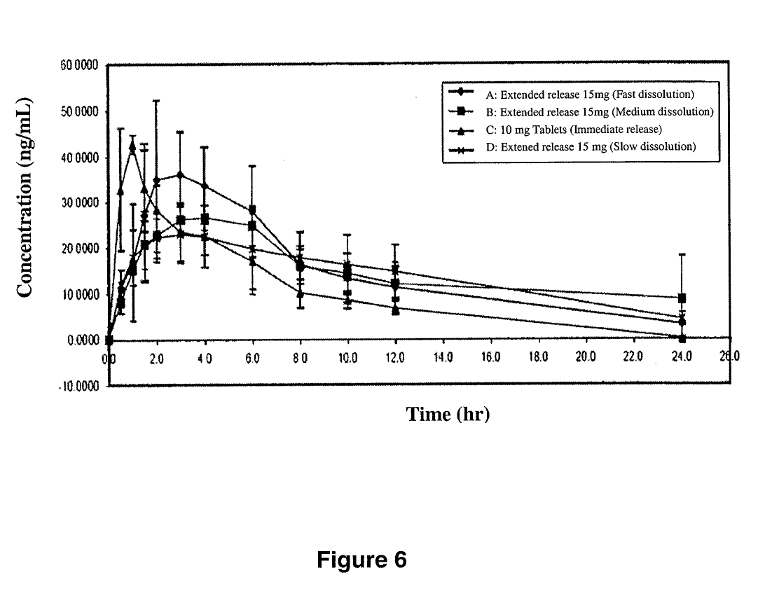

FIG. 7 shows the constitution of a tablet subject of present invention.

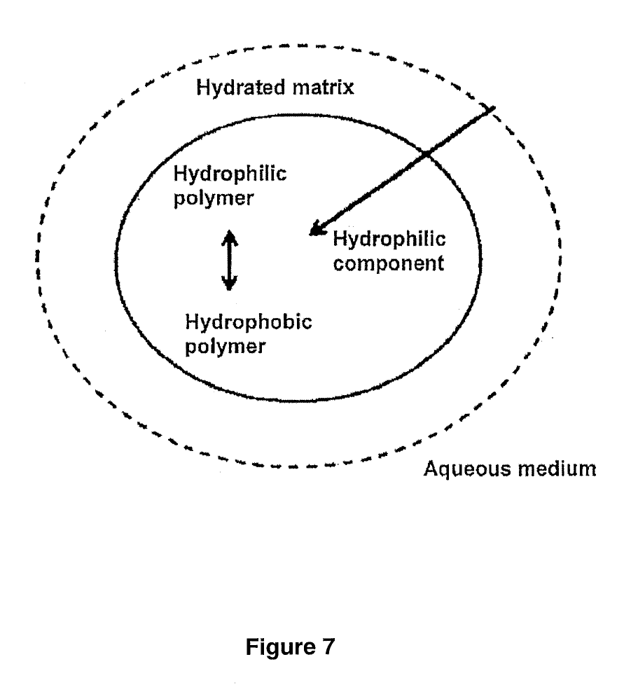

### DETAILED DESCRIPTION OF INVENTION

Present invention provides a medicament for treatment and/or prevention of gastrointestinal disorders, by delivering an effective and/or prophylactic amount of an extended release formulation containing metoclopramide hydrochloride or a pharmaceutically acceptable salt thereof, to any person in need thereof.

Present invention further provides, the use of extended release metoclopramide hydrochloride or a pharmaceutically acceptable salt thereof, for treatment and/or prevention of gastrointestinal disorders.

A procedure for formulation manufacturing is below provided, including but not limited to:

1. Drug substances and excipients are provided.

2. Drug substance and excipients are screened in order to remove lumps.

3. Components are mixed and the mixture is compressed to a 100 mg preferred weight.

4. Tablets are conditioned in packaging material.

5. Manufacturing process and used equipments are those of conventional use for manufacturing a drug product with above features.

The formulation is mainly composed of:

a) A hydrophilic polymer which is swollen by hydration upon contacting water forming a gel coating controlling a release of drug substance. The water within the matrix dissolves the drug substance and this is externally diffused through the gel coating.

The hydrophilic polymer is selected from a number of products, including: methylcellulose, hydroxyethyl cellulose, hydroxypropylcellulose and hydroxypropylmethylcellulose. On the other hand, the polymer is overhydrated on the matrix surface upon solubilization, being present a matrix wear out as a consequence of an erosion mechanism.

b) A hydrophobic polymer, showing plastic deformation under compression and tending to surround the drug substance particles, reducing the pore quantity and dimensions in the matrix structure and consequently delaying a release of drug substance. Hydrophobic polymer is selected from a plurality of products such as: ethylcellulose, glyceryl monostearate and fatty acids such as acetyl tributyl citrate.

c) An hydrophilic component, showing a synergic effect with the hydrophilic polymer and forming part of the gel coating structure providing support thereto, contributing as a consequence to the control of the drug substance release. Hydrophilic component is selected from a plurality of products such as, cross-linked binding sodium carboxymethylcellulose, cross-linked binding polyvinylpyrrolidone, sodium glycolate starch, pregelatinized starch and modified cellulose.

d) The drug substance, metoclopramide hydrochloride or a pharmaceutically acceptable salt thereof.

The formulation is designed to be delivered every 12 hours.

### Description of Formulation Component Performance

Hydrophilic matrices result from compressing a hydrophilic polymer with a drug substance of certain solubility. The hydrophilic polymer is swollen by hydration decreasing the drug substance release ratio up to a fixed or theoretically constant value. Drug substance release depends on the diffusion capability through the polymeric net, the matrix erosion capability or a combination of both processes.

In the hydrophilic matrix developed for present invention, release is controlled when the water soluble polymer is rapidly hydrated on the tablet surface to form a gel coat, which controls water penetration into said tablet. Water inside dissolves the drug substance and this is diffused through the net formed by the gel. Gel coat strength is controlled by polymer viscosity and concentration.

The water-insoluble hydrophobic polymer controls the drug substance release modifying the diffusion path size and length. Although the polymer is water insoluble, it may collect water due to the shown capability in hydrogen bridges with water.

The polymer shows plastic deformation properties under compression, tending to surround the drug substance particles, reducing the number of pores in the matrix structure contributing to drug substance release control.

The component related with water, which is swollen when being in contact with, contributes to gel formation through a synergic interaction with the water-soluble polymer, being part of the gel structure. This condition allows obtaining tablets with reproducible dissolution profiles.

### Bioavailability And Pharmacokinetics Assessment For Metoclopramide Tablets With Different Dissolution Velocities

A single-blind, parallel, single-dose, longitudinal, prospective, experimental, pilot study, in 12 healthy male subjects between 18 and 55 years was carried out in order to determine pharmacokinetics profiles, establishing and comparing bioavailability as well as assessing safety and tolerance for three 15-mg extended-release metoclopramide hydrochloride tablet formulations with several dissolution velocities and one 10-mg immediate-release metoclopramide hydrochloride tablet formulation.

They were orally administered swallowing a dose of the assigned formulation with 250 ml water, according to the treatment random assignment.

5 ml vein blood samples were collected by catheter or venopuncture on times: 0 h (pre-dosage), and later at 0.5 h, 1.0 h, 1.5 h, 2.0 h, 3.0 h, 4.0 h, 6.0 h, 8.0 h, 10.0 h, 12.0 h and 24.0 h after 10 dose. Collected plasma was kept under freezing conditions (−40° C.) until analysis. Unaltered metoclopramide in plasma samples was quantified with a validated method for High Resolution Liquid Chromatography (HPLC) with fluorescence detection.

Systemic tolerance assessment was carried out through the identification and analysis of adverse events which are present during study conduction.

### RESULTS

Table 1 reports a descriptive statistics (average, standard deviation, variation coefficient, minimum and maximum) of pharmacokinetic parameters, for each treatment group.

TABLE 1
Pharmacokinetic parameters for metoclopramide.
Arithmetic average + S.D., Variation
coefficient (%), Minimum-

| Pharmaco | | | | |
|---|---|---|---|---|
| kinetic | Treatment A | Treatment B | Treatment C | Treatment D |
| parameter | n = 3 | n = 3 | n = 3 | n = 3 |
| ABCO-t | 301.08 + 129.25 | 346.34 + 103.37 | 209.17 + 50.17 | 307.77 + 137.54 |
| (hr * ng/mL) | 42.93 | 29.85 | 23.98 | 44.69 |
| | 191.90-443.79 | 252.59-457.20 | 153.08-249.76 | 150.10-403.08 |
| AB C_{0-INF} | 345.88 ± 106.00 | 424.811 | 256.55 + 47.83 | 369.27 ± 121.12 |
| (hr * ng/mL) | 30.65 | 189.07 | 18.64 | 32.80 |
| | 275.57-467.80 | 44.51 | 201.64-289.15 | 229.80-447.99 |
| | | 274.60-637.13 | | |
| ABCM_{o-INF} | 2881.44 + 1314.21 | 5927.28 + 4648.56 | 1829.73 + 418.80 | 4428.38 + 1978.68 |
| (hr * ng/mL) | 45.61 | 78.03 | 22.89 | 44.68 |
| | 1639.03-4257.27 | 2697.29-11280.31 | 1523.80-2307.04 | 2579.74-6515.49 |
| Cmax | 40.06 ± 13.78 | 27.75 + 3.52 | 43.8613.96 | 24.16 ± 5.44 |
| (ng/ml) | 34.39 | 12.69 | 9.02 | 22.54 |
| | 25.14-52.30 | 23.69-29.92 | 40.21-48.06 | 18.41-29.23 |
| Tmax | 3.0011.00 | 4.00 ± 1.73 | 0.83 + 0.29 | 3.00 + 1.00 |
| (hr) | 33.33 | 43.30 | 34.64 | 33.33 |
| | 2.00-4.00 | 3.00-6.00 | 0.50-1.00 | 2.00-4.00 |
| Ke {hr-li | 0.1578 + 0.047 | 0.1077 + 0.0045 | 0.1494 + 0.0454 | 0.096210.0263 |
| | 329.9454 | 4.1452 | 30.3956 | 27.3796 |
| | 0.1297-0.2124 | 0.1035-0.1124 | 0.1172-0.2013 | 0.0687-0.1212 |
| t \hat{ } (hr) | 4.63 + 1.18 | 6.44 + 0.26 | 4.90 ± 1.29 | 7.61 ± 2.24 |
| | 25.54 | 4.09 | 26.40 | 29.48 |
| | 3.26-5.34 | 6.17-6.69 | 3.44-5.91 | 5.72-10.09 |
| TMR (hr) | 8.21 + 2.33 | 12.75 ± 4.31 | 7.22 + 1.53 | 11.84 ± 2.46 |
| | 28.38 | 33.80 | 21.15 | 20.77 |
| | 5.57-9.97 | 9.82-17.71 | 5.46-8.22 | 9.74-14.54 |

Wherein :
Treatment A: Extended-release metoclopramide hydrochloride tablets, 15 mg (fast dissolution).
Treatment B: Extended-release metoclopramide hydrochloride tablets, 15 mg (medium dissolution).
Treatment C: Metoelopramide hydrochloride tablets, 10 mg (immediate release).
Treatment D: Extended-release metoclopramide hydrochloride tablets, 15 mg {slow dissolution).

### Characterization of 15 mg Extended Release Metoclopramide Tablets Type

Based on pharmacokinetics parameter results shown in Table 1, and on the considerations for characterization of an extended- or controlled-release product (Blume, Gundert & R. Molly 1991. Modified release product. Wisenchaftliche Verlagegesellschaft mbH. Stutgart), the three extended-release formulations (A, B and D), show extended release kinetics, since Cmax for three products is lower than that for immediate-release product; further a Tmax delay of 0.8 h was observed in the immediate-release and 3-4 hours in extended-release products. However, clearance half-life was not apparently modified between extended-release and immediate-release products.
Metoclopramide hydrochloride profiles in plasma for extended-release formulations with fast, medium and slow dissolution show different absorption kinetics as to immediate-release profile; generally, Cmax is decreased, tmax is delayed and permanence of plasma levels higher than 10 ng/mL is observed between 3 and 12 hours for extended-release products. An apparent proportionality in metoclopramide plasma profiles is observed, regarding to in-vitro dissolution velocity.

Statistically significant differences in Tmax, Cmax and Cmax/ABCO-INF pharmacokinetic parameters were observed.

Extended-release 15 mg metoclopramide hydrochloride tablet products may be apparently characterized as “extended-release” products, since 15 Cmax is decreased, Tmax is delayed and clearance half-life is not modified regarding to an immediate release product.

During development of present extended-release metoclopramide hydrochloride pharmaceutical composition three dissolution velocities were studied, herein referred as “slow”, “medium” and “fast”, selecting among the three the best of them which is described as “medium”.

Following are examples of different optimum formulations to obtain metoclopramide hydrochloride extended release tablets to a 15 mg dose per tablet.

### Example 1

Medium dissolution velocity for extended release

| | QUANTITY |
|---|---|
| | mg/1 able |
| COMPONENT | t |
| Metoclopramide Hydrochloride | 15.00 |
| Colloidal silicon dioxide | 0.50 |
| Pregelatinized corn starch | 42.25 |
| Hydroxypropylmethylcellulose | 30.00 |
| Ethylcellulose | 10.00 |
| Magnesium stearate | 1.50 |

### EXAMPLE 2

Slow dissolution velocity for extended release tablets in a dose of 15 mg per tablet.

| COMPONENT | QUANTITY |
|---|---|
| Methoclopramide Hydrochloride | 15.00 |
| Colloidal silicon dioxide | 0.50 |
| Pregelatinized corn starch | 12.25 |
| Hydroxypropylmethylcellulose | 40.00 |
| Ethylcellulose | 30.00 |
| Magnesium stearate | 1.50 |

### EXAMPLE 3

Fast dissolution velocity for extended release tablets in a dose of 15 mg per tablet

| | QUANTITY |
|---|---|
| COMPONENT | mg/tablet |
| Metoclopramide Hydrochloride | 15.00 |
| Colloidal silicon dioxide | 0.50 |
| Pregelatinized corn starch | 72.25 |
| Hydroxypropylmethylcellulose | 10.00 |
| Ethylcellulose | 0.00 |
| Magnesium stearate | 1.50 |

1. An extended release pharmaceutical composition, a tablet of about 100 milligrams, for release into the gastrointestinal environment, comprising metoclopramide hydrochloride from about 10 to 20 milligrams by weight, from hydrophilic and hydrophobic polymers and hydrophilic components which promote water penetration within the tablet, all those from about 90 to 80 milligrams by weight, which are pharmaceutically acceptable so that when composition is orally taken, extended release is induced while keeping a bioavailability substantially equivalent to the immediate release composition.

2. Extended release pharmaceutical composition according to claim 1, characterized in that comprises hydrophilic, hydrophobic polymers as well as hydrophilic components which promote water penetration within the tablet.

3. Extended release pharmaceutical composition according to claim 1, characterized in that the hydrophilic polymer is selected from the group consisting of methylcellulose, hydroxyethylcellulose, hydroxypropylcellulose and hydroxypropylmethylcellulose.

4. Extended release pharmaceutical composition according to claim 1, characterized in that the hydrophilic component promoting water penetration inside the tablet is selected from the group consisting of crosslinked binding sodium carboxymethylcellulose, crosslinked binding polyvinylpyrrolidone/sodium glycolate starch, pregelatinized starch and modified cellulose.

5. Extended release pharmaceutical composition according to claim 1, characterized in that the extended release pharmaceutical composition in gastrointestinal environment, comprises a hydrophilic polymer, a hydrophobic polymer and a hydrophilic component in a percentage of about 90 to 80 milligrams by weight.

6. Extended release pharmaceutical composition according to claim 1, characterized in that the hydrophilic polymer is methylcellulose.

7. Extended release pharmaceutical composition according to claim 1, characterized in that the hydrophilic polymer is hydroxyethylcellulose.

8. Extended release pharmaceutical composition according to claim 1, characterized in that the hydrophilic polymer is hydroxypropylcellulose.

9. Extended release pharmaceutical composition according to claim 1, characterized in that the hydrophilic polymer is hydroxypropylmethylcellulose.

10. Extended release pharmaceutical composition according to claim 1, characterized in that the hydrophobic polymer is selected from the group consisting of ethylcellulose, glyceryl monostearate and fatty acids such as acetyl tributyl citrate.

11. Extended release pharmaceutical composition according to claim 1, characterized in that the hydrophobic polymer is ethylcellulose.

12. Extended release pharmaceutical composition according to claim 1, characterized in that the hydrophobic polymer is glyceryl monostearate.

13. Extended release pharmaceutical composition according to claim 1, characterized in that the hydrophobic polymer is a fatty acid such as acetyl tributyl citrate.

14. Extended release pharmaceutical composition according to claim 1, characterized in that the hydrophilic component is selected from the group consisting of crosslinked binding sodium carboxymethylcellulose, crosslinked binding polyvinylpyrrolidone, sodium glycolate starch, pregelatinized starch and modified cellulose.

15. Extended release pharmaceutical composition according to claim 1, characterized in that the hydrophilic component promoting water penetration inside the tablet is sodium carboxymethylcellulose.

16. Extended release pharmaceutical composition according to claim 1, characterized in that the hydrophilic component promoting water penetration inside the tablet is crosslinked binding polyvinylpyrrolidone.

17. Extended release pharmaceutical composition according to claim 1, characterized in that the hydrophilic component promoting water penetration inside the tablet is sodium glycolate starch.

18. Extended release pharmaceutical composition according to claim 1, characterized in that the hydrophilic component promoting water penetration inside the tablet is pregelatinized starch.

19. Extended release pharmaceutical composition according to claim 1, characterized in that the hydrophilic component promoting water penetration inside the tablet is modified cellulose.

20. Extended release pharmaceutical composition according to claim 1, characterized in that creates an increase in peristaltic movement amplitude in esophagus, gastric antrum and small intestine and an increase in propulsive motility from gastrointestinal content.

21. Extended release pharmaceutical composition according to claim 1, characterized in that comprises about 30 mg of metoclopramide hydrochloride or a pharmaceutically acceptable salt thereof.

22. Extended release pharmaceutical composition according to claim 1, characterized in that is administered for treatment or prevention of disorders such as: vomit, esophageal gastric reflux and nausea.

23. Extended release pharmaceutical composition according to claim 1, characterized in that the metoclopramide hydrochloride formulation or a pharmaceutically acceptable salt thereof reduce the likelihood of reaching plasma concentrations which generate extrapyramidal effects.

24. Extended release pharmaceutical composition according to claim 1, characterized in that the metoclopramide hydrochloride formulation or a pharmaceutically acceptable salt thereof show a lower frequency in administration.

25. Extended release pharmaceutical composition according to claim 1, characterized in that the metoclopramide hydrochloride formulation or a pharmaceutically acceptable salt thereof is administered every 24 hours.
